(19) United States
(12) Patent Application Publication
(10) Pub. No.: US 2026/0224108 A1
(43) Pub. Date: Aug. 6, 2026

(54) Wireless Telemetry Sensor System

(71) Applicant: SIBEL HEALTH, INC., Chicago, IL (US)

(72) Inventor: Ha Uk Chung, Niles, IL (US)

(73) Assignee: SIBEL HEALTH, INC., Chicago, IL (US)

(21) Appl. No.: 19/150,726

(22) PCT Filed: Jan. 31, 2024
(86) PCT No.: PCT/US2024/013835

§ 371 Date: Jul. 24, 2025

Related U.S. Application Data
(60) Provisional application No. 63/482,528, filed on Jan. 31, 2023.

(51) Int. Cl. A61B 5/00; A61B 5/0205; A61B 5/024; A61B 5/257; A61B 5/282; A61B 5/352

(52) U.S. Cl. CPC A61B 5/002; A61B 5/02055; A61B 5/257; A61B 5/282; A61B 5/6833; A61B 5/7267; A61B 5/7285; A61B 5/02416; A61B 5/352; A61B 2560/0443; A61B 2562/164; A61B 2562/166; A61B 2562/222

(57) ABSTRACT

The presently disclosed technique includes a multi-detector wearable medical sensor that combines biopotential, optical, temperature, acceleration, and acoustic measurement into a single device. It leverages sensor fusion and deep learning algorithms on device which allows it to directly monitor vital signs, physiological waveforms, and early warning of medical complications. One utility is the ability for the sensor to expand and adapt to several use cases. An expansion port and accessory cables allow the biopotential electrodes to step up or down for any combination of continuous 1-12 L electrocardiogrameasurements. It can also measure fetal ECG with a different accessory cable, or electroencephalogram (“EEG”) with another cable. The sensor has BLUETOOTH® Low Energy (“BLE”), Wireless Fidelity (“WIFI®)”), Fifth Generation mobile network (“5G”) Long Term Evolution Machine Type Communication (“LTE-M”), Near Field Communication (“NFC”), and Ultra Wideband (“UWB”) radios. The sensor can also charge and communicate over Universal Serial Bus (“USB”).

### CROSS-REFERENCE TO RELATED APPLICATION(S)

This application claims priority to U.S. Provisional Patent Application No. 63/482,528 filed Jan. 31, 2023, the contents of which is incorporated herein by reference for all purposes, including the purpose of priority.

### TECHNICAL FIELD

The present disclosure related to systems for monitoring physiological parameters with a wireless network for comprehensive patient monitoring in both hospital and home settings.

### BACKGROUND

This section of this document introduces information about and/or from the art that may provide context for or be related to the subject matter described herein and/or claimed below. It provides background information to facilitate a better understanding of the various aspects of the present invention. This is a discussion of “related” art. That such art is related in no way implies that it is also “prior” art. The related art may or may not be prior art. The discussion in this section of this document is to be read in this light, and not as admissions of prior art.

Monitoring vital signs are key clinical practice in hospital settings. Telemetry monitors allow continuous measurement of vital signs while patients can freely move outside of bedside. However, the existing technology remains a heavy and bulky physical embodiment. In addition, measuring all vital signs typically require connections to multiple sensors through wires or cables that further limits the usability of the system. The embedded screen size in a conventional telemetry monitor also provides low visibility. Therefore, the invention targets to overcome limitations with the conventional telemetry monitor system.

### SUMMARY

The presently disclosed technique includes a multi-detector wearable medical sensor that combines biopotential, optical, temperature, acceleration, and acoustic measurement into a single device. It leverages sophisticated sensor fusion and deep learning algorithms on device which allows it to directly monitor vital signs, physiological waveforms, and early warning of medical complications. One utility is the ability for the sensor to expand and adapt to several use cases. An expansion port and accessory cables allow the biopotential electrodes to step up or down for any combination of continuous 1-12 L electrocardiogrameasurements. It can also measure fetal ECG with a different accessory cable, or electroencephalogram (“EEG”) with another cable. The sensor has BLUETOOTH® Low Energy (“BLE”), Wireless Fidelity (“WIFI®”), Fifth Generation mobile network (“5G”) Long Term Evolution Machine Type Communication (“LTE-M”), Near Field Communication (“NFC”), and Ultra Wideband (“UWB”) radios. The sensor can also charge and communicate over Universal Serial Bus (“USB”).

In one embodiment, a medical sensor worn on a patient's body comprises a plurality of detectors and a processing system. The plurality of detectors measure two or more of biopotential, optical, temperature, acceleration, and acoustic measurements from the patient's body and transmitting the measurements. The detectors are disposed within the processing system. The processing system is programmed to interchangeably collect data from among the data transmitted by the detectors, analyze physiological characteristics of the patient's condition from the collected data, and wirelessly transmit the analyzed physiological characteristics.

In another embodiment, a method for monitoring a patient's physical condition comprises: applying a conformable medical sensor to the patient's body, the sensor including a plurality of different types of detectors disposed within a processing system configured to interchangeably collect measurements from the detectors; sensing two or more of biopotential, optical, temperature, acceleration, and acoustic measurements of the patient through the detectors; analyzing, by the processing system, a plurality of physiological characteristics of the patient's condition from the sensed measurements received by the processing system; and wirelessly transmitting the analyzed physiological characteristics from the processing system.

In yet another embodiment, a sensor worn on a patient's body, the sensor comprises a physiological signal monitoring sensor programmed to collect: a time-synchronized electrocardiogra (“ECG”), bio-impedance (“Bioz”), and photoplethysmography (“PPG”) data to calculate a heart rate (“HR”), a respiration rate (“RR”), and a temperature of the patient; a time synchronized pulse arrival time (“PAT”) between the ECG data and the PPG data to estimate a blood pressure reading for the patient; and a PPG signal for measuring both a chest reflectance oxygen saturation (“SpO2”) continuously and a spot check SpO2 on a finger of the patient.

In this embodiment, the sensor includes a pair of thermistors, a motion sensor, a MEMs microphone, an onboard BLUETOOTH® radio module, a haptic interface, and a processing system. The pair of thermistors measure a skin temperature and an ambient temperature to estimate core body temperature. The motion sensor is programmed to measure time-dependent motion signals modulated by the patient's respiration and seismocardiography (“SCG”) . The MEMS microphone measures continuous heart and lung sounds. Note that other embodiments employing a microphone may use microphone technologies other than MEMs. The onboard BLUETOOTH® radio module connects to a mobile gateway device to transmit and display data into hospital monitoring tools. The haptic interface programmed to generate a haptic response. The processing system includes a microcontroller (“MCU”) and is programmed to process and transmit the raw data of ECG, Bioz, temperatures, PPGs, and SCGs to a gateway device.

In still another embodiment, a body-worn medical sensor comprises a configurable number a low-noise and multi-lead AFE. The configurable number of electrodes that can be connected to a consumable biopotential adhesive layer for continuously measuring clinical grade multi-lead ECG signals. The low-noise and multi-lead AFE for measuring clinical grade ECG when mounted on chest, fECG when mounted on intrapartum patient's belly, and EEG when mounted on a baby's head.

In another embodiment, a body-worn medical sensor for wireless telemetry operation comprises: an onboard BLUETOOTH® radio module communicating with patient-worn smart gateway devices such as smartphone and smartwatch in a distance of BLUETOOTH® communication range with the sensor to allow telemetry operation for patients inside of hospital and transport settings; a wireless communication of the sensor data via BLUETOOTH® to tablet PC as a clinical monitor that further communicates to a central monitoring station in hospital; and an onboard WIFI® radio module to directly transmit and communicate with a central monitoring station in hospital.

In still another embodiment, a body-wearable medical sensor is substantially as shown and described.

In yet another embodiment, a method for monitoring a patient substantially as shown and described.

In still another embodiment, a system for monitoring a patient substantially as shown and described.

The above presents a simplified summary of the invention as claimed below in order to provide a basic understanding of some aspects of the invention. This summary is not an exhaustive overview of the invention. It is not intended to identify key or critical elements of the invention or to delineate the scope of the invention. Its sole purpose is to present some concepts in a simplified form as a prelude to the more detailed description that is discussed later.

### BRIEF DESCRIPTION OF THE DRAWINGS

In the drawings, like reference numbers generally indicate identical, functionally similar, and/or structurally similar elements.

FIG. 1 is a rendering image of one embodiment of a body-worn medical sensor including a processing system attached to a consumable adhesive layer.

FIG. 2A is a rendering image showing a sensor with pogo pins that can be guided into the consumable adhesive layer with female pogo pins to make electrical connection.

FIG. 2B shows an example of when the sensor is fully connected to the consumable adhesive layer.

FIG. 3 is a block diagram of one particular embodiment of the processing system.

FIG. 4A is a block diagram of one particular embodiment of the processing system of FIG. 1 and FIG. 2A-FIG. 2B and FIG. 4B is an exemplary printed circuit board (“PCB”).

FIG. 5 shows an example of the body-worn medical sensor mounted on patient's chest to collect continuous 8 channel ECG.

FIGS. 6A-FIG. 6D show another form factor of the body-worn medical sensor including an all-in-one integrated analog-front end (“AFE”) for measuring ECG, photoplethysmography (“PPG”), and bio-impedance (“Bioz”) with dockable pins to one or more medical sensor bodies.

FIG. 7 shows another mounting for measuring fetal ECG on a pregnant mother.

FIG. 8 shows another mounting for measuring infant electroencephalogram (“EEG”).

FIG. 9 is a photograph of a patient wearing both a patch sensor on their chest and an oximeter sensor on their finger, with both sensors including physiological sensors and a haptic interface.

FIG. 10 shows the wireless sensor network for bi-directional data exchange via BLUETOOTH® between a) the body-worn medical sensor and a pair of another body-worn medical sensors, b) the body-worn medical sensor and standard hospital equipment, c) the pair of another body-worn medical sensor and standard hospital equipment.

FIG. 11 shows the direct communication and data exchange between the body-worn medical sensor and central monitoring station through WIFI®.

FIG. 12A shows representative signals of ECG or EKG, PPG, EEG, seismocardiography (“SCG”), respiration, and temperature.

FIG. 12B shows a representative raw EEG signal superimposed with a gold standard data.

While the disclosed subject matter is susceptible to various modifications and alternative forms, the drawings illustrate specific implementations described in detail by way of example. It should be understood, however, that the description herein of specific examples is not intended to limit that which is claimed to the particular forms disclosed, but on the contrary, the intention is to cover all modifications, equivalents, and alternatives falling within the spirit and scope of the appended claims.

### DETAILED DESCRIPTION OF THE INVENTION

In view of the foregoing, it would be beneficial to have a body-worn medical sensor that can measure all vital signs concurrently. In one aspect, the sensor measures electrocardiogramar calculating heart rate (“HR”) and respiration rate (“RR”), photoplethysmography (“PPG”) for calculating oxygen saturation (“SpO2”) and RR, bio-impedance (“Bioz”) for calculating RR and tidal volume estimation, skin temperature, core body temperature, and pulse arrival time (“PAT”) for estimating blood pressure (“BP”).

In one embodiment, the body-worn medical sensor has an integrated analog-front end (“AFE”) that measures ECG, BioZ, and PPG synchronously. The synchronized timing difference between a R-peak of the ECG signal and a valley of the PPG signal then calculates PAT.

In other embodiments, the body-worn medical sensor has a 3-axial accelerometer. The accelerometer can measure time-dependent motion signals in x-, y-, and z-axis. The 3-axial information can estimate body position, fall detection, and gait.

In related embodiments, the body-worn medical sensor is mounted on the chest area. The z-axis of the accelerator can then measure the seismocardiography (“SCG”) that captures the chest wall movement. The SCG data can estimate RR and pre-ejection period (herein “PEP”).

In other embodiments, the body-worn medical sensor has a microelectromechanical (herein “MEMS”) microphone. The microphone is programmed to transform the acoustic sound pressure into a time-dependent digital output of continuous heart sound and lung sound, when mounted on the chest.

In other embodiments, the sensor includes a BLUETOOTH® 5 enabled system-on-a-chip (BT SoC) and sensing units for monitoring physiological parameters. The microcontroller unit (MCU) in the BT SoC configures the sensing units and processes data, then the BT SoC transmits the data through BLUETOOTH® communications. The sensor also receives the data from other BLUETOOTH® enabled devices to trigger alarming events both visually and physically.

In other embodiments, the sensor has an on-board Wi-Fi radio module that supports IEEE 802.11n or higher. It allows the direct communication to hospital network through Wi-Fi from the sensor.

In other embodiments, the sensor contains a haptic interface that can be either a vibratory motor, mechanical buzzer, a device that delivers an electric current to the patient, a light emitting diode (“LED”), a piezoelectric device, and a device that emits an acoustic sound.

In other embodiments, the sensor comprises of a minimum of a pair of electrodes for sensing electrical signal of patient's ECG signal and inject electrical current into the patient for impedance measurement of patient.

In related embodiments, the sensor has electrical, dockable pins, such as pogo pins, that can be connected to a consumable adhesive layer comprising conductive electrodes and skin adhesive. The consumable adhesive layer can be configured with any number of electrodes to allow the sensor to measure not only single-lead ECG but also up to 12-lead ECG for clinical ECG measurement.

In another aspect, the invention provides a body-worn medical sensor that includes: 1) a respiration sensor comprising a pair of electrodes that measure a time-dependent electrical signal modulated by the patient's respiration; 2) a haptic interface that generates a haptic response; and 3) a processing system that receives the motion signals from the motion sensor and the time-dependent electrical signal from the respiration sensor, and operates computer code that: i) processes the motion signals to determine a first signal related to a presence or absence of a breathing; ii) processes the electrical signals to determine a second signal related to the patient's posture change that may cause the patient to fall from the bed; and iii) controls the haptic interface to generate the haptic response for waking up the patient and warning patients for falling, respectively.

Turning now to the drawings, illustrative examples of the subject matter claimed below are disclosed. In the interest of clarity, not all features of an actual implementation are described for every example in this specification. It will be appreciated that in the development of any such actual implementation, numerous implementation-specific decisions may be made to achieve the developers' specific goals, such as compliance with system-related and business-related constraints, which will vary from one implementation to another. Moreover, it will be appreciated that such a development effort, even if complex and time-consuming, would be a routine undertaking for those of ordinary skill in the art having the benefit of this disclosure.

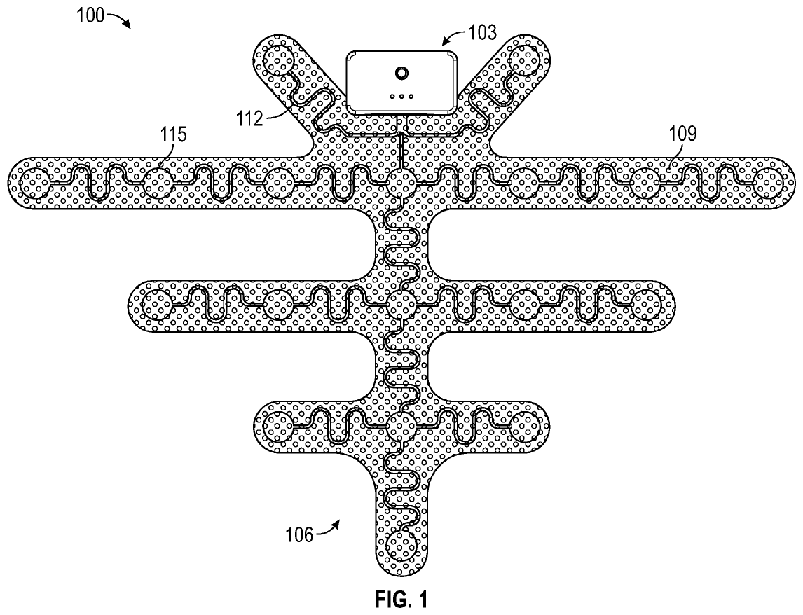

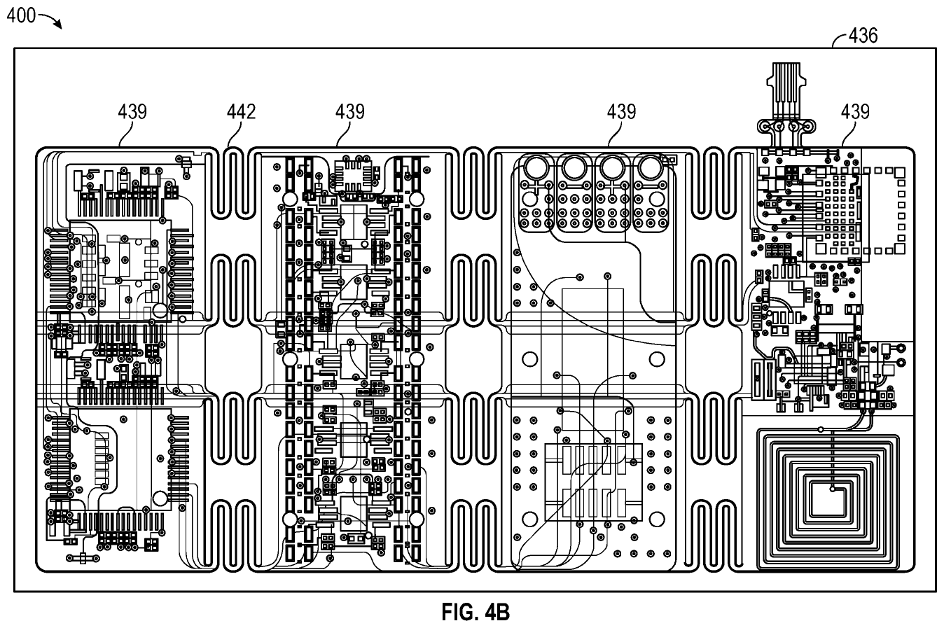

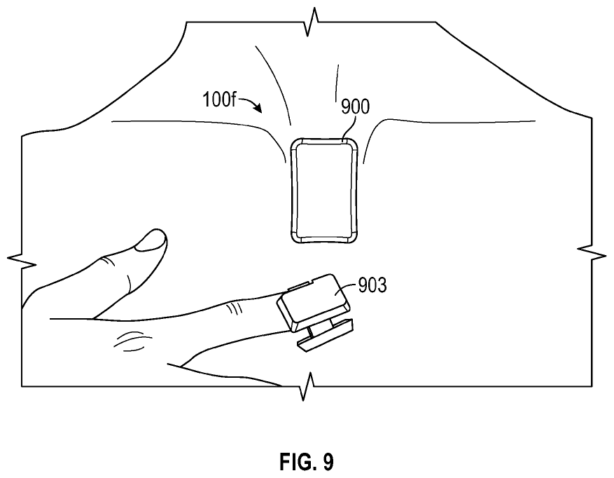

FIG. 1 is a rendering image of one embodiment of a body-worn medical sensor 100 including a processing system 103 attached to an optional consumable adhesive layer 106. Some embodiments may omit the consumable adhesive layer 106, and in these embodiments the body-worn medical sensor may comprise only the processing system 103, in which are disposed a plurality of detectors not separately shown. One such embodiment is discussed relative to FIG. 4A-FIG. 4B. Another embodiment omitting the consumable adhesive layer 106 and including a separate detector disposed without the processing system 103 is discussed relative to FIG. 9.

The consumable adhesive layer 106 includes a conformable material 109 fabricated of a flexible, conformable material in which a plurality of wires 112 (only one indicated) are disposed. The consumable adhesive layer 106 also includes a plurality of electrodes 115 (only one indicated) disposed thereon or therein electrically connected to the wires 112 of the consumable adhesive layer 106.

As mentioned above, the processing system 103 includes a plurality of detectors not separately shown. The detectors, in operation, interchangeably sense two or more of biopotential, optical, temperature, acceleration, and acoustic measurements. The processing system 103 then wirelessly transmits the measurements.

As used herein, the term “interchangeably” means that the processing system 103 may be programmed to process and analyze data from selected, but not necessarily all, the detectors and, in some embodiments electrodes. The processing system 103 may be, for example, programmed differently for each use to collect and analyze measurements from different sensors to determine different physiological conditions in each use. In this sense, the medical sensor 100 may be said to be “configured to” monitor certain physiological characteristics of the patient by programming the processing system 103 to collect and process data from selected detectors. One way to program or “configure” the body-worn medical sensor disclosed herein is discussed below.

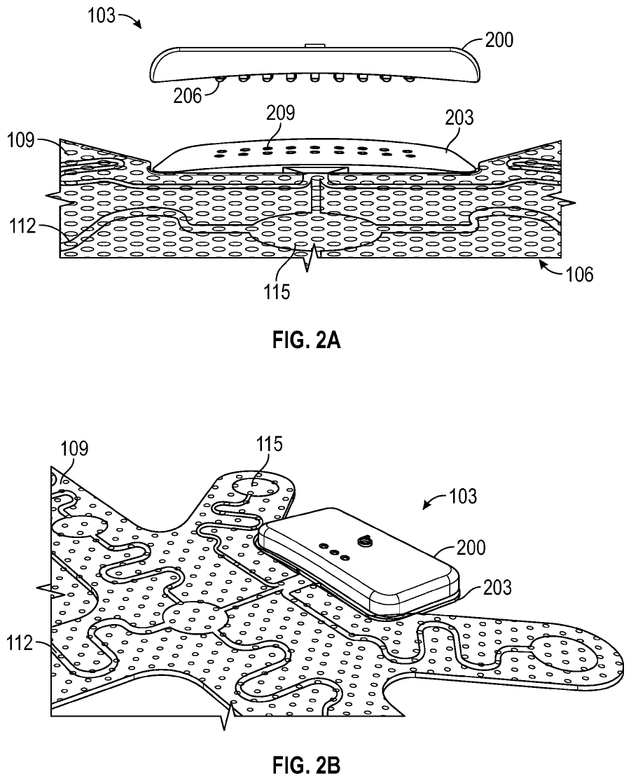

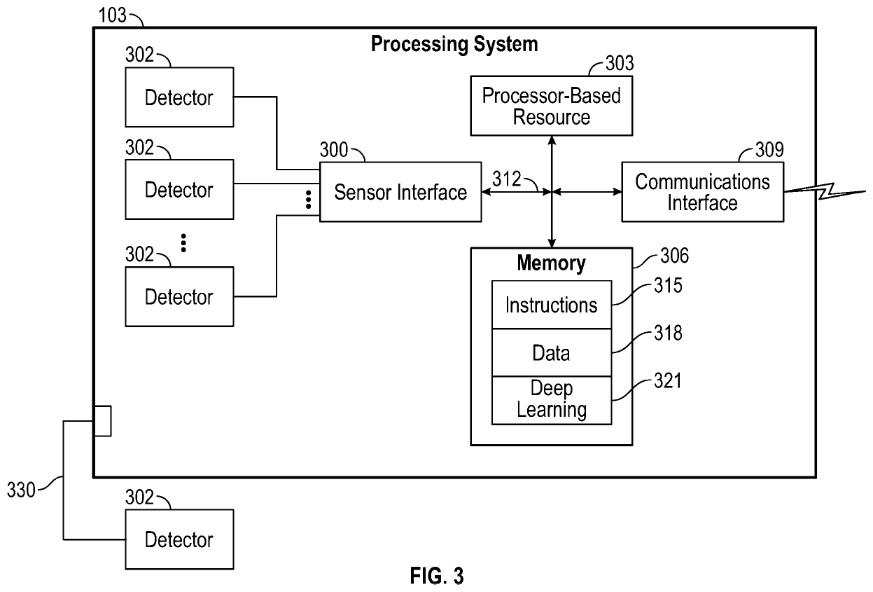

Returning to the embodiment of FIG. 1, the processing system 103 is structurally engaged with the consumable adhesive layer 106 and electrically connected to the electrodes 115 via the wires 112 of the consumable adhesive layer 106 as shown in FIG. 2A-FIG. 2B. In the illustrated embodiment, the processing system 103 comprises an upper housing 200 and a lower mounting pad 203. As used herein, directional terms such as “top”, “upper”, “lower”, and “bottom” are defined relative to the patient's body, with the “bottom”, “lower”, and similar terms indicating the closest or more proximal to the patient's body and the “top”, “upper” and similar terms indicating the most far or distal from the patient's body.

The lower mounting pad 203 is removably affixed to the conformable material 109, for example by adherence, of the consumable adhesive layer 106. The upper housing 200 contains the electronics, not shown in FIG. 2A-FIG. 2B, of the processing system 103 as discussed further below. The upper housing 200 is mounted by lowering the upper housing 200 onto the lower mounting pad 203. A plurality of electrical, dockable pins 206, only one indicated, extending from the upper housing 200 are inserted into a plurality of sockets 209, only one indicated, in the lower mounting pad 203. In the illustrated embodiment, the dockable pins 206 are pogo pins. As the upper housing 200 continues to be lowered, the pogo pins 206 extend through the lower mounting pad 203 in a manner not shown, to electrically connect with the electrical wires 112. The processing system 103 is thereby structurally engaged with the consumable adhesive layer 106 and electrically connected to the electrodes 115 via the wires 112.

The electronics, and thereby the processing system 103, are programmed to collect data transmitted by the electrodes 115. The processing system 103 then analyzes physiological characteristics of the patient's condition from the collected data. The results of the analysis are then wirelessly transmitted to, for instance, a patient monitor, a central monitoring station, or a mobile device such as a phone, tablet, or laptop, or some other destination not shown.

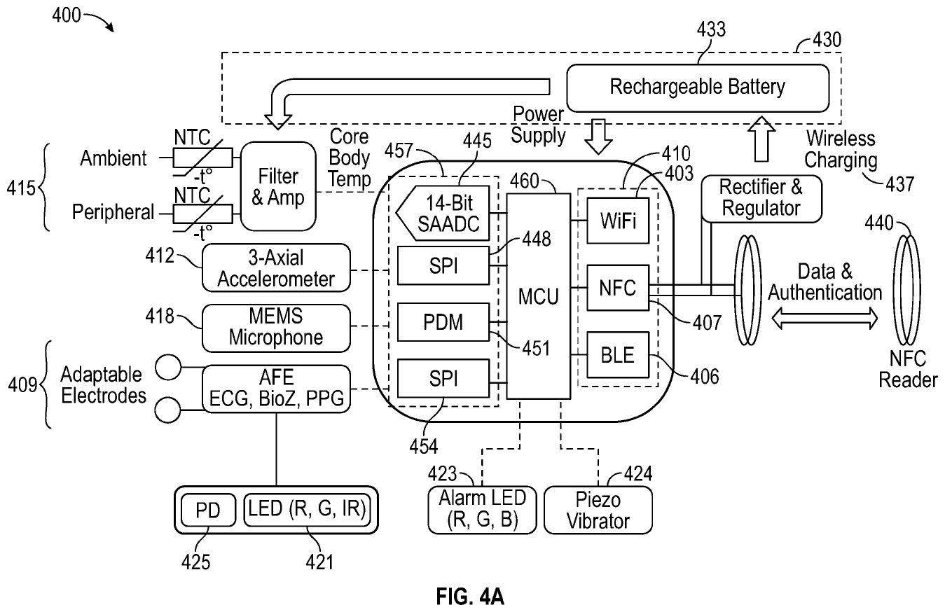

FIG. 3 is a block diagram of one particular embodiment of the processing system 103. In general, it is contemplated by the present disclosure that the processing system 103 includes electronic components and/or electronic computing devices operable to receive, transmit, process, store, and/or manage patient data and information associated performing the functions of the system as described herein, which encompasses any suitable processing device adapted to perform computing tasks consistent with the execution of computer-readable instructions stored in a memory or a computer-readable recording medium. This includes the electronic components to collect data transmitted by the detectors, analyze physiological characteristics of the patient's condition from the collected data, and wirelessly transmit the analyzed physiological characteristics.

The processing system 103 is a processing system and includes, as shown in FIG. 3, a sensor interface 300, a processor-based resource 303, a memory 306, and a communications interface 309 communicating over a bus system 312. Those in the art having the benefit of this disclosure will appreciate that the claimed subject matter admits wide variation for these aspects of the processing system 103. For example, implementation of the sensor interface 300 and the communications interface 309 will depend on the types of detectors and/or electrodes employed and the communications protocols employed. Similarly, those in the art having the benefit of this disclosure will appreciate that some aspects of the processing system 103 may be separated into separate components or combined into fewer components.

The sensor interface 300 is used to connect via the bus system 312 to the detectors 302 and, when present, electrodes 115 for gathering types of physiological data from the patient. The sensor interface 300 may be implemented in hardware, software, or combinations thereof. The sensor interface 300 may include a data acquisition circuit further including amplifying and filtering circuity as well as analog-to-digital (“A/D”) circuity that converts the analog signal to a digital signal using amplification, filtering, and A/D conversion methods.

The processor-based resource 303 may be any electronic chip or chipset built around one or more processor cores, whether a single-core processor or a multi-core processor. Examples of such include, but are not limited to, a central processing unit (“CPU”), a hardware microprocessor, a processing system, a microcontroller, a digital signal processor (“DSP”), etc. In a chipset, the processor-based resource 303 may also include any kind of co-processor built around a processor core, such as a math co-processor or a graphics co-processor. However, the processor-based resource 303 is not limited to processors built around a processor core. The processor-based resource 303 may also be, in some implementations, an application specific integrated circuit (“ASIC”), a field programmable gate array (“FPGA”), and erasable programmable read-only memory (“EPROM”), or an electrically erasable programmable memory (“EEPROM”). The processor-based resource may be any kind of processing device capable of executing any type of instructions, algorithms, or software for controlling the operation and performing the functions of the processing system 103.

The memory 306 may be one or more single memory devices or memory structures and may be on-chip or off-chip relative to the processor-based resource 303. The memory 306 may include, without limitation, one or more of a random-access memory (“RAM”), a memory buffer, a hard drive, a database, an erasable programmable read only memory (“EPROM”), an electrically erasable programmable read only memory (“EEPROM”), a read only memory (“ROM”), a flash memory, hard disk, various layers of memory hierarchy, or any other non-transitory computer readable medium. The memory 306 may be used to store any type of instructions 315 and patient data 318 associated with algorithms, processes, or operations 321 for controlling the general functions and operations of the processing system 103. Those in the art having the benefit of this disclosure will appreciate that the processing system 103 may be considered to be “programmed to” perform the functionalities disclosed herein in the sense that the processor-based resource 303 is programmed by the content of the memory 306.

In the illustrated embodiments, the algorithms 321 include deep learning algorithms running on deep learning modules to analyze physiological characteristics of the patient's condition from the collected data. Deep learning models running on-device allow real-time analysis of multiple data streams, gaining greater insight than analyzing data streams separately using traditional clinical algorithms. Deep learning models are created by passing raw signals through layers of interconnected weights. In the disclosed embodiments, the raw signals are the signals generated by the electrodes 115 containing the data indicative of selected, predetermined physiological characteristics of the patient's condition. This data may also include, for example, data received from the detectors 302. During training of the model, each layer's weights are iteratively updated to meet a global objective, for example, estimating patient health.

The global objective allows the model to learn different levels of feature representation as data flows through the layers of the network. For example, once optimized on the global objective, early layers will often learn transformations related to simpler signal patterns, while higher level layers will learn semantic representations of the data. This training process allows superior performance by generating novel features from signal combinations without a priori knowing what signal attributes will be important for achieving the global objective.

It is contemplated by the present disclosure that the communications interface 309 includes adequate electronics, components, programming, etc. needed to wirelessly transmit the results of the analysis off-sensor as described elsewhere herein. The communication interface 309 may include circuity for receiving data from and sending data to one or more devices using, for example, a WIFI® connection, a cellular network connection, and/or a BLUETOOTH® connection, etc., none of which are separately shown in FIG. 3. The wireless communication connections can allow, for example, patient and hospital information, alerts, and physiological data to be transmitted in real-time within a hospital wireless communications network (e.g., WIFI®) as well as allow for patient and hospital information, alerts, and physiological data to be transmitted in real-time to other devices. Those in the art will appreciate that still other wireless communications protocols and/or standards may be used in addition to, or in lieu of, those shown in other embodiments.

It is also contemplated by the present disclosure that the communication connections established by the processor-based resource 303 through the communications interface 309 permits communications over other types of wireless networks using alternate hospital wireless communications. Such alternate hospital wireless communications may include, without limitation, wireless medical telemetry service (“WMTS”), which can operate at specified frequencies (e.g., 1.4 GHz). Other wireless communication connections can include wireless connections that operate in accordance with, but are not limited to, IEEE802.11 protocol, a Radio Frequency For Consumer Electronics (“RF4CE”) protocol, ZIGBEE® protocol, and/or IEEE802.15.4 protocol.

The BLUETOOTH® or WIFI® connection can also be used to transfer of data to a nearby device (e.g., a phone, a tablet, or a patient monitor) or to a more remote locations (e.g., a central monitoring station) for review of data and the results of analysis. The receiving device, or “external gateway”, may include a display/GUI including a GUI for displaying patient information, physiological data or measured data, measurement schedules, alerts or alarms for the patient, clinicians and/or caregiver's information. The communications interface 309 provides these and other communications capability to the processing system 103.

The embodiment of FIG. 3 also includes an optional cable 330, detector 302, and port 333. One or more such cables 330 may be used for one or more detectors 302 disposed without the processing system 103 through one or more ports 333 to communicate data to the processor-based resource 303. For instance, a set of ECG leads (not separately shown) may be deployed on the patient's body and interfaced with the processing system 103 in this manner. Thus, in various embodiments, the medical sensor may be deployed as only the processing system 103, the processing system 103 in conjunction with a consumable adhesive layer 106, the processing system 103 in conjunction with one or more cables 330, and/or the processing system 103 in conjunction with both the consumable adhesive layer 106 and one or more cables 330.

FIG. 4A is a functional block diagram of a body-worn medical sensor 400. In one embodiment, the medical sensor 400 comprises of a system-on-a-chip (herein “SoC”) with internal WIFI® and BLUETOOTH® radio modules 403, 406 that configures the peripheral modules of an AFE with configurable electrodes 409 for ECG, BioZ, and PPG, a high-frequency 3-axis accelerometer 412 for SCG and movement features, thermometers 415 based on negative temperature coefficient (“NTC”) thermistors with filters and amplifier to measure both skin and core body temperature, a MEMS microphone 418, alarming light emitting diodes (“LEDs”) 421, 423, a photodiode (“PD”) 425, haptic (e.g., piezo) vibrator 427, and a power management circuit 430. The power management circuit 430 includes rechargeable battery 433 and wireless charging circuit 433 supporting a near-field communication (“NFC”) wireless charging standard (“WLC”). The PD is the optical detector that may be used for PPG detection to calculate SpO2.

Those in the art having the benefit of this disclosure will appreciate that the WIFI®, NFC, and BLUETOOTH® radio modules 403, 406, 407 in FIG. 4A comprise a communications interface 410. Similarly, the configurable electrodes 409 for ECG, BioZ, and PPG, a high-frequency 3-axis accelerometer 412 for SCG and movement features, thermometers 415 based on negative temperature coefficient (“NTC”) thermistors with filters and amplifier to measure both skin and core body temperature, a MEMS microphone 418, alarming light emitting diodes (“LEDs”) 421, 423, a photodiode (“PD”) 425, haptic (e.g., piezo) vibrator 427, comprise the electrodes 115 in FIG. 1 in this particular embodiment. The 14-bit Successive approximation analog-to-digital (“SAADC”) circuit 445, serial peripheral interface (“SPI”) 448, pulse digital modulator (“PDM”) 451, and SPI 454 comprise a sensor interface 457.

Referring to FIG. 4B, these modules are populated on flexible PCB 436 that connects multiple islands 439 through flexible and stretchable serpentine interconnects 442 (only one indicated). Note that the number and shape of the flexible PCB 436, the islands 439, and the serpentine interconnects will be implementation specific dependent upon design considerations. These design considerations may include factors such as the number and placement of the detectors, the identity of the detectors, the design of the consumable adhesive layer, etc.

Referring again to FIG. 4A, the microcontroller (“MCU”) 460 inside of the SoC configures the peripheral modules via digital communication protocol such as serial peripheral interface (“SPI”), the inter-integrated circuit (“I2C”), improved inter integrated circuit (“13C”), for modules that are operating in digital mode. The MCU 460 also samples the analog signals such as temperature data output through an analog-digital converter (“ADC”), either internal ADC inside of the SoC or independent one. Those in the art having the benefit of this disclosure will appreciate that the MCU 460 will include one or more processor cores (not separately shown) as well as on-chip memory (also not separately shown). The one or more processor cores are programmed with the instructions and algorithms stored in the on-chip memory. The on-chip memory will also store such data as is used to execute those instructions and algorithms.

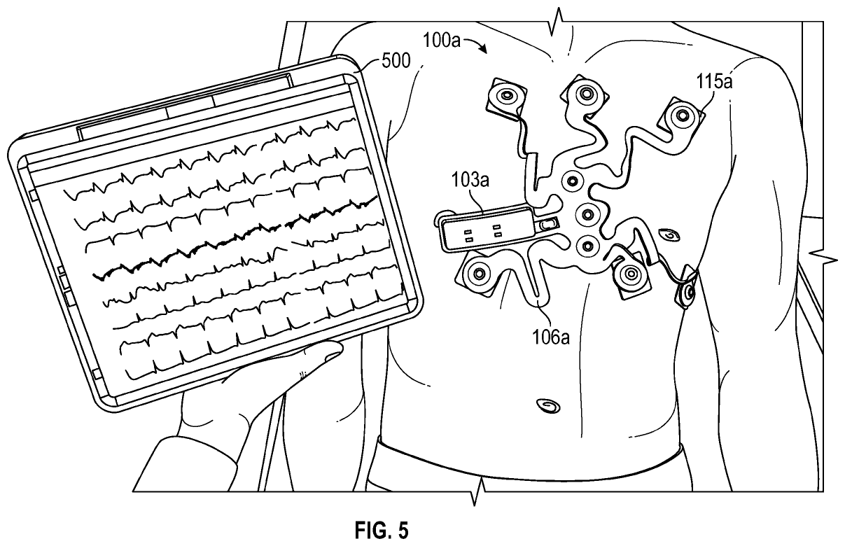

Referring to FIG. 4A, the BLUETOOTH® module 406 transmits processed data and signals from the sensor to an external gateway represented by its antenna 440 supporting BLUETOOTH®. In one embodiment, the gateway can be a smartphone with a custom developed Android or iOS application installed. In another embodiment, the gateway can be a tablet personal computer (“PC”) or a smartwatch with similar operating characteristics to the smartphone. The embodiment of FIG. 5, for instance, shows an external gateway in the form of a tablet 500

Still referring to FIG. 4A, the sensor configures the notification or haptic interface, neither of which is separately shown, that can be either a vibratory motor, mechanical buzzer, a device that delivers an electric current to the patient, a Light Emitting Diode (“LED”) such as a tricolor LED (e.g. red, green, blue) 423, a piezoelectric device 424, and/or a device (not shown) that emits an acoustic sound.

In one aspect, the body-worn medical sensor will transmit multiple physiological signals via BLUETOOTH® (ECG, PPG, skin temperature, core body temperature, mechano-acoustic signals, and microphone signals) to be displayed real-time on a mobile device and central monitoring station. The sensor first would measure the five vital signs: 1) HR derived from ECG, PPG, and SCG, 2) RR derived from ECG, BioZ, PPG, and acoustically by microphone, 3) SpO2 and perfusion index (“PI”) derived from PPG on chest, 4) body temperature from the temperature flux by the two NTC thermistors, 5) a continuous surrogate measurement of BP based on the timing difference of the pulse between ECG and PPG called PAT. The sensor further measures unique parameters: 1) heart sounds by SCG and microphone, 2) fall count, step count, body position by the accelerometer, 3) vocalizations by both the microphone and accelerometer when mounted around suprasternal notch, and 4) total sleep time.

In one embodiment, the body-worn medical sensor is encapsulated in a soft housing material having a low effective moduli with connectable pinouts such as in the form of a pogo pins for making electrical connections to a consumable adhesive layer as is shown in FIG. 1 and FIG. 2A-2B. The combination of soft material and flexible PCB renders a mechanical embodiment of thin and light weight. Such a design allows conformable contact to skin when the sensor is attached to patient.

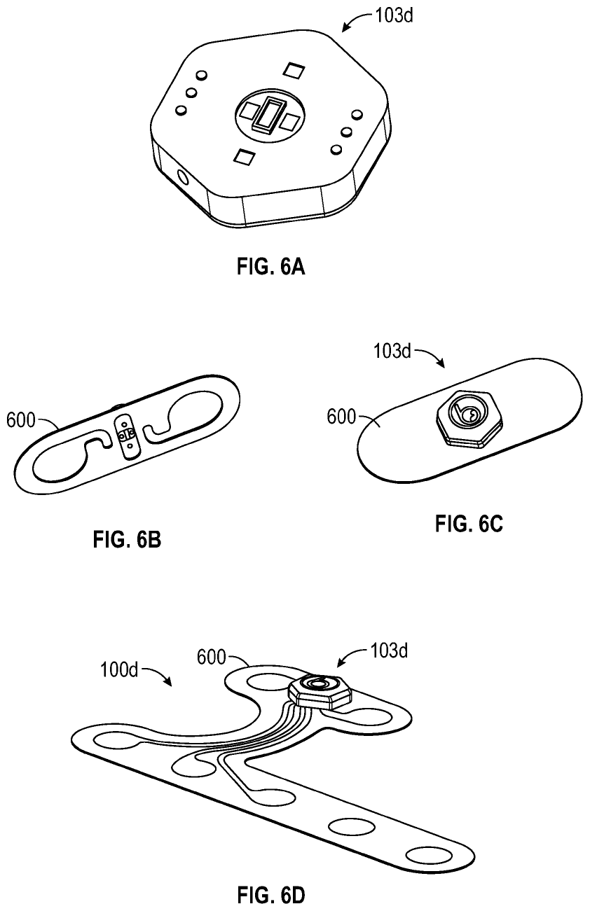

In addition, the consumable adhesive layer comprising of a soft base material having low effective moduli such as silicone gel with low effective moduli and conductive gel such as biocompatible hydrogel at the interface of electrode to skin is dockable to the body-worn medical sensor through pogo pin connections and provides the conformable contact to patient's skin. In another embodiment, the consumable adhesive layer can have multiple shapes including but not limited to ones shown in FIG. 1, FIG. 5, and FIG. 6A-FIG. 6D. Although each of these designs for the consumable adhesive layer differs, they can each be described as having a “trunk” with “branches” extending therefrom.

In one aspect, the body-worn medical sensor measures multi-lead physiological signal concurrently, especially up to 12-lead clinical ECG with a configurable consumable adhesive layer by adding an extra multi-lead biopotential AFE. FIG. 5 represents one embodiment of a medical sensor 100a for continuously streaming 8 channel ECG. Note the shape and position of the processing system 103a, the application of the consumable adhesive layer 106a to the patient's body, and the positioning of the electrodes 115a (only one indicated). Also note that, in this embodiment, only the processing system 103a and the electrodes 115a are adhered to the patient's body. In one embodiment, the configurable consumable adhesive layer 106a can be used to collect ECG as little as single-lead, but as many as to collect 12-lead ECG signals. Note further the display of the detected waveforms on the external gateway, i.e., the tablet 500.

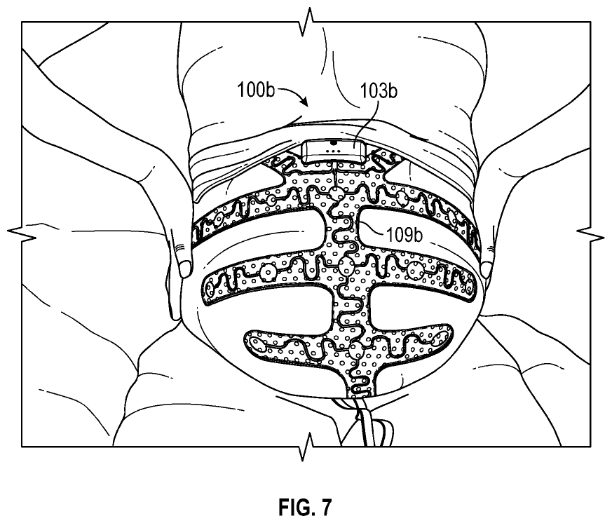

In one embodiment shown in FIG. 7, a body-worn medical sensor 100b and the configurable consumable adhesive layer 106b mounts on an intrapartum patient's belly to collect fetal ECG (“fECG”) with 8 channel measurements. In related embodiment, the body-worn medical sensor 100b has arrays of the biopotential AFEs to collect channels of fECG data in the multiple of 8. In related embodiment, the body-worn medical sensor 100b also can be used to measure maternal vital signs in parallel.

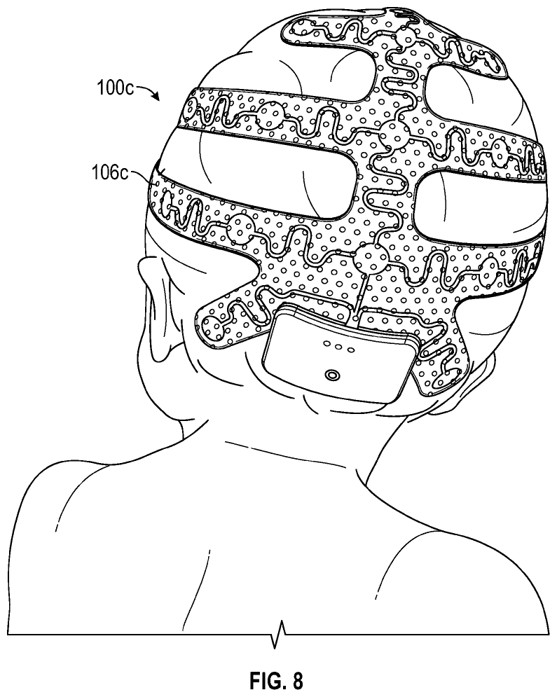

In related embodiment shown in FIG. 8, the body-worn medical sensor 100c and the consumable adhesive layer 106c mounts on a baby's head to collect continuous EEG signal. Similar to fECG, the body-worn medical sensor can be programmed to collect multiple EEG channels data in multiples of 8 by adding arrays of AFEs.

In related embodiment, the consumable adhesive layer may be encapsulated with soft silicone housing material to avoid contact to skin except the conductive electrode areas for signal capturing. In another embodiment, the consumable adhesive layer may be used with a conventional ECG electrode by clicking them into holes in the consumable adhesive layer. One such embodiment is shown in FIG. 5.

A haptic motor connects to the PCB board as mentioned above. The haptic motor can take several forms, all of which exhibit vibratory action (i.e. ‘buzzing’) when activated. One form is an ‘eccentric rotating mass’ component that, when driven with a time-dependent analog or digital signal controlled by the embedded microprocessor, causes the haptic motor to rotate in a specific direction to cause a vibration that is then felt by the patient. A second form is a ‘linear resonant actuator’ that typically features a hockey puck-type shape and vibrates in a similar manner in response to the analog or digital signal. A third type is a ‘piezoelectric module’ that rapidly expands and contracts in response to the driving signal (typically a time-dependent analog voltage), thereby causing it to vibrate. Other types of haptic motors or actuators, particularly those that can be easily mounted to the PCB, can also be used for this application.

In one embodiment shown in FIG. 6A-FIG. 6D, the body-worn medical sensor 100d, has a reflectance mode PPG 600 on the bottom of the processing system 103d to collect a continuous chest SpO2, while measuring other parameters including ECG, Bioz, temperatures, and PATs concurrently. The reflectance operation can also be used as a spot check tool for SpO2, PI, and PR. FIG. 6C is a single lead embodiment and FIG. 6D is a multi-lead embodiment.

As described above relative to FIG. 4A, a rechargeable Li-polymer battery powers the electronics on the PCB. An inductive coil (not shown in the figure) imprinted or integrated onto the PCB charges the battery when it is exposed to an electromagnetic field. The charging can be compatible to NFC WLC standard. In one embodiment, the body-worn medical sensor can be charged on compatible off the shelf charger supporting the same NFC WLC standard. Alternatively, the PCB may include a port, such as a USB port, that connects to a power source to recharge the battery.

In related embodiments, the haptic interface can be coupled to vital signs and parameters such as values of systolic BP (“SYS”) and diastolic BP (“DIA”) determined by PAT, and other methodologies. For example, the haptic interface could automatically be initiated if the patient's blood pressure values trended beyond pre-determined limits that may be harmful to the patient. Such events could occur, for example, during periods of exercise, stress, etc.

Referring to FIG. 9, the sensor shown in FIG. 1 can be divided into other embodiments. In FIG. 9, the first portion 900 (“Chest”) of a body-worn medical sensor 103f is applied directly to the patient's chest, a location where accurate measurement of HR and RR is most likely to be made. As described above, this parameter can be measured with two or more different sensing modalities, e.g., an accelerometer, ECG, and/or impedance circuitry, the latter of which is described in more detail below. The second portion 903 (“Limb”) is positioned in patient's limb where accurate measurement of SpO2 and Pl is likely to be made.

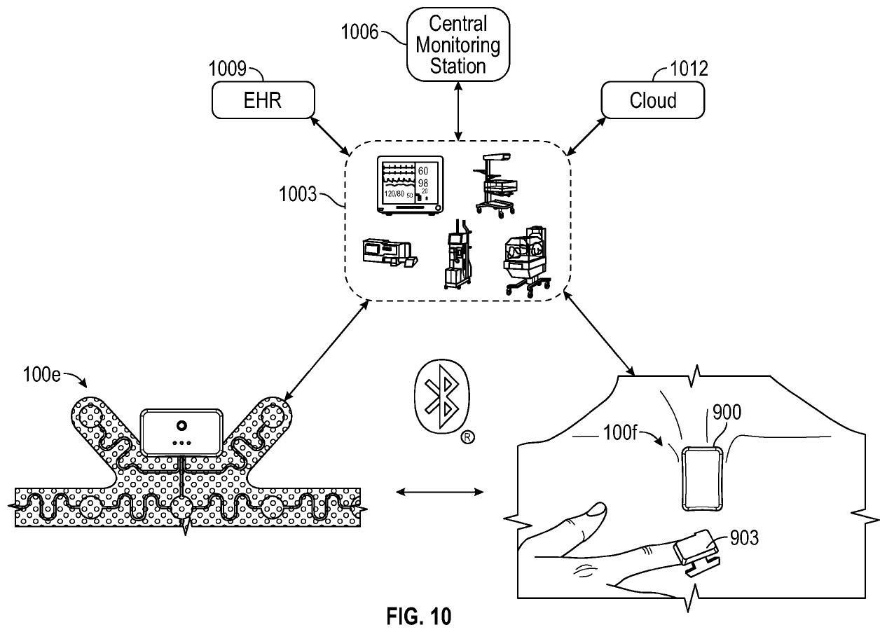

Referring to FIG. 10, the body-worn medical sensor 100e can communicate with another pair 1000 of Chest and Limb sensors 900, 903, described above, via BLUETOOTH® for data exchange such as measured parameters and the timing information. Either the body-worn medical sensor 100e or the body-worn medical sensor 100f can serve as the master device recording the timing information. Then, either sensor can also communicate with hospital equipment 1003 capable of BLUETOOTH® communication to transmit and display the parameters on the equipment such as hospital monitor, incubators, and others. In addition, the transmitted data can be further transmitted to central monitoring station 1006, electronic health record (“EHR”) 1009, or other cloud storage place 1012.

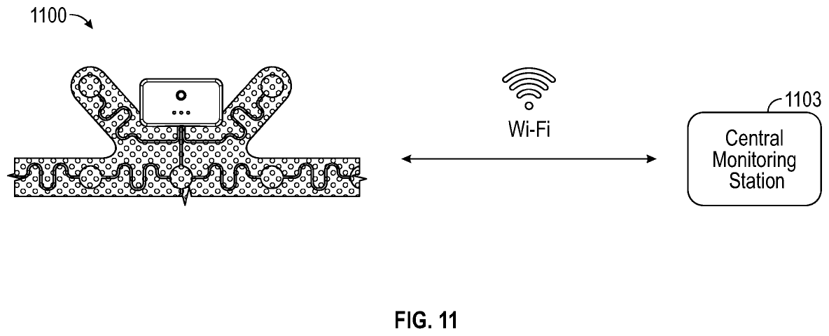

In other embodiment shown in FIG. 11, a body-worn medical sensor 1100 has an on-board WIFI® module (not separately shown) that can directly communicate to a central monitoring station 1103 at a hospital or other medical care facility that allows telemetry operation. Patients wearing the sensor 1100 can freely move without having any gateway devices attached to them, while measuring all the vital signs and advanced features described above in a single sensor.

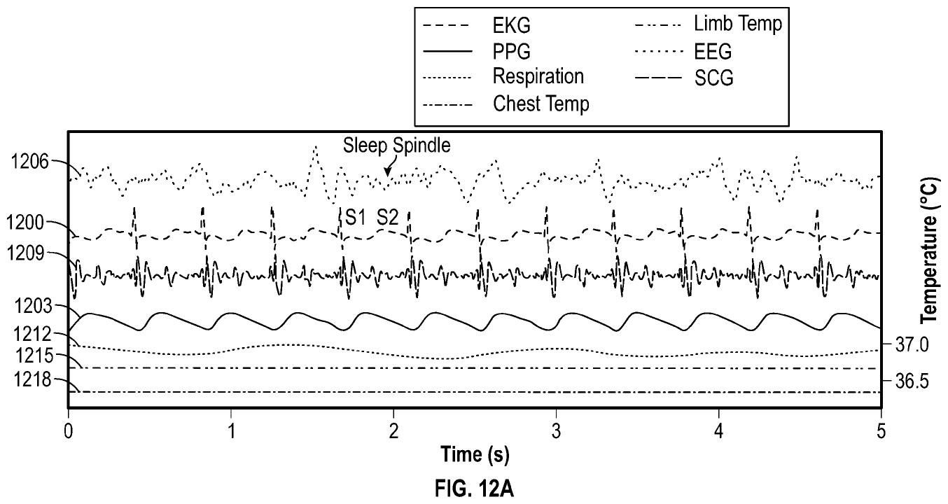

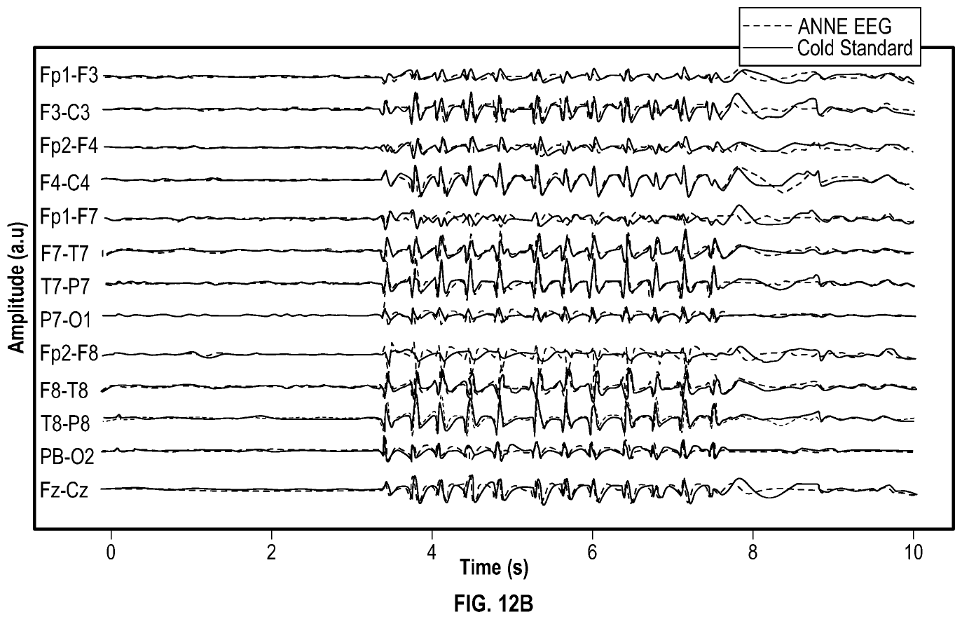

Referring to FIG. 12A, the plot shows a representative signals of ECG or EKG 1200, PPG 1203, EEG 1206, SCG 1209, respiration 1212, limb temperature 1215, and chest temperature 1218 that can be collected from embodiments of the body-worn medical sensor described above. FIG. 12B shows a representative signal of EEG measured on a baby's head, superimposed with a gold standard data, in the embodiment described above as EEG measurement sensor.

In other embodiments, the sensor described herein could count physiological events that can be easily measured with an accelerometer, like coughing, sneezing, and aspiration, and then apply a haptic interface to the patient when these events exceed a predetermined level.

In other embodiments, the haptic interface can be timed to decrease the amount of drooling experienced by the patient. The body-worn medical sensor can also include a microphone which measures sounds emitted by the patient, and can be used to decrease the patient's slurring or drive them to speak louder if their speech is too light.

Accordingly, in a first embodiment, a medical sensor worn on a patient's body comprises a plurality of detectors and a processing system. The plurality of detectors measure two or more of biopotential, optical, temperature, acceleration, and acoustic measurements from the patient's body and transmitting the measurements. The detectors are disposed within the processing system. The processing system is programmed to interchangeably collect data from among the data transmitted by the detectors, analyze physiological characteristics of the patient's condition from the collected data, and wirelessly transmit the analyzed physiological characteristics.

In a second embodiment, the medical sensor of the first embodiment further comprises a consumable adhesive layer to which the processing system is structurally engaged. The consumable adhesive layer includes a flexible, conformable material defining the consumable adhesive layer; at least one electrical wire disposed in the consumable adhesive layer, and a plurality of electrodes disposed in the consumable adhesive layer. The electrodes measure electrical activity of the patient's body and transmit the measurements thereof to the processing system, the electrodes electrically connected to the processing system via the at least one electrical wire. The processing system is programmed to interchangeably collect data transmitted by the electrodes.

In a third embodiment, the medical sensor of the second embodiment further comprises one or more additional detectors disposed without the processing system. The additional detectors communicate with the processing system over one or more cables. The processing system is programmed to interchangeably collect data transmitted by the one or more additional detectors.

In a fourth embodiment, the medical sensor of the first embodiment further comprises one or more additional detectors disposed without the processing system. The one or more additional detectors communicate with the processing system over one or more cables. The processing system is programmed to interchangeably collect data transmitted by the one or more additional detectors.

In a fifth embodiment, analyzing physiological characteristics in the medical sensor of the first embodiment includes employing deep learning algorithms.

In a sixth embodiment, in the medical sensor of the first embodiment, the processing system includes a sensor interface collecting measurements transmitted by the detectors; a memory on which resides instructions and algorithms; a processor-based resource executing the instructions residing on the memory and employing the algorithms to analyze physiological characteristics of the patient's condition from the collected data; and a communications interface by which the processor-based resource wirelessly transmits the analyzed physiological characteristics.

In a seventh embodiment, in the medical sensor of the sixth embodiment, the processing system comprises a System on a Chip (“SoC”).

In an eighth embodiment, in the medical sensor of the sixth embodiment, the processor-based resource is a microcontroller.

In a ninth embodiment, in the medical sensor of the sixth embodiment, the processing system comprises a flexible printed circuit board on which are mounted or fabricated a plurality of electronics.

In a tenth embodiment, in the medical sensor of the first embodiment, wherein the processing system comprises a System on a Chip.

In an eleventh embodiment, in the medical sensor of the tenth embodiment, the System on a Chip includes a microcontroller.

In a twelfth embodiment, in the medical sensor of the first embodiment, the processing system comprises a flexible printed circuit board on which are mounted or fabricated a plurality of electronics.

In a thirteenth embodiment, in the medical sensor of the first embodiment, two or more of the measurements are time-synchronized.

In a fourteenth embodiment, in the medical sensor of the thirteenth embodiment, two or more of the measurements are time-synchronized by the difference between an R-peak of an electrocardiogramal and a valley of a photoplethysmography signal.

In a fifteenth embodiment, in the medical sensor of the first embodiment, the physiological characteristics of the patient's condition include two or more of blood oxygen saturation, heart rate, respiration rate, body temperature, tidal volume, patient temperature, pulse arrival time, pre-ejection period, or combinations thereof.

In a sixteenth embodiment, in the medical sensor of the first embodiment, the measurements include an electrocardiogrameasurement, a bio-impedance measurement, or a photoplethysmography measurement, or a seismocardiography measurement, or an inertial measurement, or an aural measurement, or combinations thereof.

In a seventeenth embodiment, in the medical sensor of the first embodiment, the detectors include an electrocardiogrampedance detector, a photoplethysmography detector, a seismocardiography detector, an accelerometer, a microphone, or combinations thereof.

In an eighteenth embodiment, in the medical sensor of the first embodiment, the processing system includes an integrated analog front end.

In a nineteenth embodiment, in the medical sensor of the second embodiment, the processing system is structurally engaged with the consumable adhesive layer and electrically connected to the wires of the consumable adhesive layer by a plurality of pogo pins.

In a twentieth embodiment, the medical sensor of the first embodiment further comprises a detector disposed without the processing system and wirelessly communicating with the processing system.

In a twenty-first embodiment, a method for monitoring a patient's physical condition comprises: applying a conformable medical sensor to the patient's body, the sensor including a plurality of different types of detectors disposed within a processing system configured to interchangeably collect measurements from the detectors; sensing two or more of biopotential, optical, temperature, acceleration, and acoustic measurements of the patient through the detectors; analyzing, by the processing system, a plurality of physiological characteristics of the patient's condition from the sensed measurements received by the processing system; and wirelessly transmitting the analyzed physiological characteristics from the processing system.

In a twenty-second embodiment, the method of the twenty-first embodiment further comprises: applying a conformable consumable adhesive layer to the patient's body, the consumable adhesive layer including a plurality of electrodes positioned at predetermined locations on the patient's body and operably connected to a conformable processing system via a plurality of electrical wires disposed in the consumable adhesive layer; and transmitting the sensed measurement from the electrodes to the processing system via the electrical wires;

In a twenty-third embodiment, the method of the twenty-first embodiment further comprises: disposing a detector without the processing system on the patient's body to sense an additional measurement; and wirelessly transmitting the additional measurement to the processing system.

In a twenty-fourth embodiment, in the method of the twenty-first embodiment, two or more of the sensed measurement are time-synchronized.

In a twenty-fifth embodiment, in the method of the twenty-first embodiment, the two or more sensed measurements are time-synchronized by the difference between an R-peak of an electrocardiogramal and a valley of a photoplethysmography signal.

In a twenty-sixth embodiment, in the method of the twenty-first embodiment, the physiological characteristics of the patient's condition include two or more of a blood oxygen saturation, a heart rate, a respiration rate, a body temperature, a tidal volume, a patient temperature, a pulse arrival time, a pre-ejection period, or combinations thereof.

In a twenty-seventh embodiment, in the method of the twenty-first embodiment, the sensed measurements include an electrocardiogrameasurement, or a bio-impedance measurement, or a photoplethysmography measurement, or a seismocardiography measurement, or an inertial measurement, or an aural measurement, or combinations thereof.

In a twenty-eighth embodiment, in the method of the twenty-first embodiment, the detectors include an electrocardiogramic, a bio-impedance detector, a photoplethysmography detector, a seismocardiography detector, an accelerometer, a microelectromechanical microphone, or combinations thereof.

In a twenty-ninth embodiment, a sensor worn on a patient's body, the sensor comprises a physiological signal monitoring sensor programmed to collect: a time-synchronized electrocardiogra (“ECG”), bio-impedance (“Bioz”), and photoplethysmography (“PPG”) data to calculate a heart rate (“HR”), a respiration rate (“RR”), and a temperature of the patient; a time synchronized pulse arrival time (“PAT”) between the ECG data and the PPG data to estimate a blood pressure reading for the patient; and a PPG signal for measuring both a chest reflectance oxygen saturation (“SpO2”) continuously and a spot check SpO2 on a finger of the patient.

The sensor of the twenty-ninth embodiment includes a pair of thermistors, a motion sensor, a MEMs microphone, an onboard BLUETOOTH® radio module, a haptic interface, and a processing system. The pair of thermistors measure a skin temperature and an ambient temperature to estimate core body temperature. The motion sensor is programmed to measure time-dependent motion signals modulated by the patient's respiration and seismocardiography (“SCG”). The MEMS microphone measures continuous heart and lung sounds. The onboard BLUETOOTH® radio module connects to a mobile gateway device to transmit and display data into hospital monitoring tools. The haptic interface programmed to generate a haptic response. The processing system includes a microcontroller (“MCU”) and is programmed to process and transmit the raw data of ECG, Bioz, temperatures, PPGs, and SCGs to a gateway device.

In a thirtieth embodiment, in the sensor of the twenty-ninth embodiment, the physiological signal monitoring sensor comprises a single module.

In a thirty-first embodiment, in sensor of the twenty-ninth embodiment, the physiological signal monitoring sensor comprises a plurality of sub-modules.

In a thirty-second embodiment, a body-worn medical sensor comprises a configurable number a low-noise and multi-lead AFE. The configurable number of electrodes that can be connected to a consumable biopotential adhesive layer for continuously measuring clinical grade multi-lead ECG signals. The low-noise and multi-lead AFE for measuring clinical grade ECG when mounted on chest, fECG when mounted on intrapartum patient's belly, and EEG when mounted on a baby's head.

In a thirty-third embodiment, a body-worn medical sensor for wireless telemetry operation comprises: an onboard BLUETOOTH® radio module communicating with patient-worn smart gateway devices such as smartphone and smartwatch in a distance of BLUETOOTH® communication range with the sensor to allow telemetry operation for patients inside of hospital and transport settings; a wireless communication of the sensor data via BLUETOOTH® to tablet PC as a clinical monitor that further communicates to a central monitoring station in hospital; and an onboard WIFI® radio module to directly transmit and communicate with a central monitoring station in hospital.

In a thirty-fourth embodiment, a body-wearable medical sensor is substantially as shown and described.

In a thirty-fifth embodiment, a method for monitoring a patient substantially as shown and described.

In a thirty-sixth embodiment, a system for monitoring a patient substantially as shown and described.

The expressions such as “include” and “may include” which may be used in the present disclosure denote the presence of the disclosed functions, operations, and constituent elements, and do not limit the presence of one or more additional functions, operations, and constituent elements. In the present disclosure, terms such as “include” and/or “have”, may be construed to denote a certain characteristic, number, operation, constituent element, component or a combination thereof, but should not be construed to exclude the existence of or a possibility of the addition of one or more other characteristics, numbers, operations, constituent elements, components or combinations thereof.

As used herein, the article “a” is intended to have its ordinary meaning in the patent arts, namely “one or more.” Herein, the term “about” when applied to a value generally means within the tolerance range of the equipment used to produce the value, or in some examples, means plus or minus 10%, or plus or minus 5%, or plus or minus 1%, unless otherwise expressly specified. Further, herein the term “substantially” as used herein means a majority, or almost all, or all, or an amount with a range of about 51% to about 100%, for example. Moreover, examples herein are intended to be illustrative only and are presented for discussion purposes and not by way of limitation.

As used herein, to “provide” an item means to have possession of and/or control over the item. This may include, for example, forming (or assembling) some or all of the item from its constituent materials and/or, obtaining possession of and/or control over an already-formed item.

Unless otherwise defined, all terms including technical and/or scientific terms used herein have the same meaning as commonly understood by one of ordinary skill in the art to which the present disclosure pertains. In addition, unless otherwise defined, all terms defined in generally used dictionaries may not be overly interpreted. In the following, details are set forth to provide a more thorough explanation of the embodiments. However, it will be apparent to those skilled in the art that embodiments may be practiced without these specific details. In other instances, well-known structures and devices are shown in block diagram form or in a schematic view rather than in detail in order to avoid obscuring the embodiments. In addition, features of the different embodiments described hereinafter may be combined with each other, unless specifically noted otherwise. For example, variations or modifications described with respect to one of the embodiments may also be applicable to other embodiments unless noted to the contrary.

Further, equivalent or like elements or elements with equivalent or like functionality are denoted in the following description with equivalent or like reference numerals. As the same or functionally equivalent elements are given the same reference numbers in the figures, a repeated description for elements provided with the same reference numbers may be omitted. Hence, descriptions provided for elements having the same or like reference numbers are mutually exchangeable.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

In the present disclosure, expressions including ordinal numbers, such as “first”, “second”, and/or the like, may modify various elements. However, such elements are not limited by the above expressions. For example, the above expressions do not limit the sequence and/or importance of the elements. The above expressions are used merely for the purpose of distinguishing an element from the other elements. For example, a first box and a second box indicate different boxes, although both are boxes. For further example, a first element could be termed a second element, and similarly, a second element could also be termed a first element without departing from the scope of the present disclosure.

A sensor refers to a component which converts a physical quantity to be measured to an electric signal, for example, a current signal or a voltage signal. The physical quantity may for example comprise electromagnetic radiation (e.g., photons of infrared or visible light), a magnetic field, an electric field, a pressure, a force, a temperature, a current, or a voltage, but is not limited thereto.

ECG signal processing, as used herein, refers to, without limitation, manipulating an analog signal in such a way that the signal meets the requirements of a next stage for further processing. ECG signal processing may include converting between analog and digital realms (e.g., via an analog-to-digital or digital-to-analog converter), amplification, filtering, converting, biasing, range matching, isolation and any other processes required to make a sensor output suitable for processing.

Use of the phrases “capable of,” “capable to,” “operable to,” or “programmed to” in one or more embodiments, refers to some apparatus, logic, hardware, and/or element designed in such a way to enable the use of the apparatus, logic, hardware, and/or element in a specified manner. Use of the phrase “exceed” in one or more embodiments, indicates that a measured value could be higher than a pre-determined threshold (e.g., an upper threshold), or lower than a pre-determined threshold (e.g., a lower threshold). When a pre-determined threshold range (defined by an upper threshold and a lower threshold) is used, the use of the phrase “exceed” in one or more embodiments could also indicate a measured value is outside the pre-determined threshold range (e.g., higher than the upper threshold or lower than the lower threshold). The subject matter of the present disclosure is provided as examples of apparatus, systems, methods, circuits, and programs for performing the features described in the present disclosure. However, further features or variations are contemplated in addition to the features described above. It is contemplated that the implementation of the components and functions of the present disclosure can be done with any newly arising technology that may replace any of the above-implemented technologies.

The detailed description is made with reference to the accompanying drawings and is provided to assist in a comprehensive understanding of various example embodiments of the present disclosure. Changes may be made in the function and arrangement of elements discussed without departing from the spirit and scope of the disclosure. Various embodiments may omit, substitute, or add various procedures or components as appropriate. For instance, features described with respect to certain embodiments may be combined in other embodiments. In addition, descriptions of well-known functions and constructions may be omitted for clarity and conciseness. Accordingly, those of ordinary skill in the art will recognize that various changes and modifications of the examples described herein can be made without departing from the spirit and scope of the present disclosure.

Various modifications to the disclosure will therefore be readily apparent to those skilled in the art, and the generic principles defined herein may be applied to other variations without departing from the spirit or scope of the present disclosure. Throughout the present disclosure the terms “example,” “examples,” or “exemplary” indicate examples or instances and do not imply or require any preference for the noted examples. Thus, the present disclosure is not to be limited to the examples and designs described herein but is to be accorded the widest scope consistent with the principles and novel features disclosed.

1. A medical sensor worn on a patient's body, the medical sensor comprising:
a plurality of detectors measuring two or more of biopotential, optical, temperature, acceleration, and acoustic measurements from the patient's body and transmitting the measurements; and
a processing system in which the detectors are disposed, the processing system programmed to interchangeably collect data from among the data transmitted by the detectors, analyze physiological characteristics of the patient's condition from the collected data, and wirelessly transmit the analyzed physiological characteristics.

2. The medical sensor of claim 1,
further comprising a consumable adhesive layer to which the processing system is structurally engaged, the consumable adhesive layer including:
a flexible, conformable material defining the consumable adhesive layer;
at least one electrical wire disposed in the consumable adhesive layer,
a plurality of electrodes disposed in the consumable adhesive layer to measure electrical activity of the patient's body and transmit the measurements thereof to the processing system, the electrodes electrically connected to the processing system via the at least one electrical wire,
wherein the processing system is programmed to interchangeably collect data transmitted by the electrodes.

3. The medical sensor of claim 2,
further comprising one or more additional detectors disposed without the processing system and communicating with the processing system over one or more cables,
wherein the processing system is programmed to interchangeably collect data transmitted by the one or more additional detectors.

4. The medical sensor of claim 1,
further comprising one or more additional detectors disposed without the processing system and communicating with the processing system over one or more cables,
wherein the processing system is programmed to interchangeably collect data transmitted by the one or more additional detectors.

5. The medical sensor of claim 1, wherein analyzing physiological characteristics includes employing deep learning algorithms.

6. The medical sensor of claim 1, wherein the processing system includes:
a sensor interface collecting measurements transmitted by the detectors;
a memory on which resides instructions and algorithms;
a processor-based resource executing the instructions residing on the memory and employing the algorithms to analyze physiological characteristics of the patient's condition from the collected data; and
a communications interface by which the processor-based resource wirelessly transmits the analyzed physiological characteristics.

7-9. (canceled)

10. The medical sensor of claim 1, wherein the processing system comprises a System on a Chip.

11. (canceled)

12. The medical sensor of claim 1, wherein the processing system comprises a flexible printed circuit board on which are mounted or fabricated a plurality of electronics.

13. The medical sensor of claim 1, wherein two or more of the measurements are time-synchronized.

14. The medical sensor of claim 13, wherein two or more of the measurements are time-synchronized by the difference between an R-peak of an electrocardiogramal and a valley of a photoplethysmography signal.

15. (canceled)

16. The medical sensor of claim 1, wherein the measurements include an electrocardiogrameasurement, a bio-impedance measurement, or a photoplethysmography measurement, or a seismocardiography measurement, or an inertial measurement, or an aural measurement, or combinations thereof.

17. The medical sensor of claim 1, wherein the detectors include an electrocardiogrampedance detector, a photoplethysmography detector, a seismocardiography detector, an accelerometer, a microphone, or combinations thereof.

18. (canceled)

19. The medical sensor of claim 2, wherein the processing system is structurally engaged with the consumable adhesive layer and electrically connected to the wires of the consumable adhesive layer by a plurality of pogo pins.

20. The medical sensor of claim 1, further comprising a detector disposed without the processing system and wirelessly communicating with the processing system.

21. A method for monitoring a patient's physical condition, the method comprising:
applying a conformable medical sensor to the patient's body, the sensor including a plurality of different types of detectors disposed within a processing system configured to interchangeably collect measurements from the detectors;
sensing two or more of biopotential, optical, temperature, acceleration, and acoustic measurements of the patient through the detectors;
analyzing, by the processing system, a plurality of physiological characteristics of the patient's condition from the sensed measurements received by the processing system; and
wirelessly transmitting the analyzed physiological characteristics from the processing system.

22. The method of claim 21, further comprising:
applying a conformable consumable adhesive layer to the patient's body, the consumable adhesive layer including a plurality of electrodes positioned at predetermined locations on the patient's body and operably connected to a conformable processing system via a plurality of electrical wires disposed in the consumable adhesive layer; and
transmitting the sensed measurement from the electrodes to the processing system via the electrical wires;

23. The method of claim 21, further comprising:
disposing a detector without the processing system on the patient's body to sense an additional measurement; and
wirelessly transmitting the additional measurement to the processing system.

24. The method of claim 21, wherein two or more of the sensed measurement are time-synchronized.

25. The method of claim 21, wherein the two or more sensed measurements are time-synchronized by the difference between an R-peak of an electrocardiogra signal and a valley of a photoplethysmography signal.

26-31. (canceled)

32. A body-worn medical sensor comprising:
a configurable number of electrodes that can be connected to a consumable biopotential adhesive layer for continuously measuring clinical grade multi-lead ECG signals;
a low-noise and multi-lead AFE for measuring clinical grade ECG when mounted on chest, fECG when mounted on intrapartum patient's belly, and EEG when mounted on a baby's head.

33-36. (canceled)
